(19) United States
(12) Patent Application Publication
(10) Pub. No.: US 2022/0118015 A1
(43) Pub. Date: Apr. 21, 2022

(54) MODIFIED IL-12 T CELL THERAPY FOR THE TREATMENT OF CANCER

(71) Applicant: BOARD OF REGENTS, THE UNIVERSITY OF TEXAS SYSTEM, Austin, TX (US)

(72) Inventors: Shulin LI, Houston, TX (US); Jiemiao HU, Houston, TX (US); Xueqing XIA, Houston, TX (US); Qingnan ZHO, Houston, TX (US)

(73) Assignee: BOARD OF REGENTS, THE UNIVERSITY OF TEXAS SYSTEM, Austin, TX (US)

(21) Appl. No.: 17/310,328

(22) PCT Filed: Jan. 31, 2020
(86) PCT No.: PCT/US2020/016016

§ 371 Date: Jul. 28, 2021

Related U.S. Application Data
(60) Provisional application No. 62/800,136, filed on Feb. 1, 2019.

(51) Int. Cl. A61K 35/17; C07K 14/54; C07K 14/71; A61K 31/675; A61K 31/519; A61K 31/513; A61K 31/704; A61K 31/475; A61K 33/243; A61K 31/7068; A61K 31/255; A61P 35/00

(52) U.S. Cl. CPC A61K 35/17; C07K 14/5434; C07K 14/71; A61K 31/675; A61K 31/519; A61K 31/513; C07K 2319/30; A61K 31/475; A61K 33/243; A61K 31/7068; A61K 31/255; A61P 35/00; A61K 31/704

(57) ABSTRACT

Provided herein are chimeric antigen receptor (CAR)-like constructs comprising tumor-targeted and membrane-anchored IL-12. Also provided herein are T cells expressing the CAR-like IL-12 construct. Further, methods of treating cancer comprising administering T cells expressing CAR-like IL-12 are provided herein.

This application claims the benefit of U.S. Provisional Patent Application No. 62/800,136, filed Feb. 1, 2019, which is incorporated herein by reference in its entirety.

### BACKGROUND

### 1. Field

The present invention relates generally to the fields of immunology and medicine. More particularly, it concerns modified IL-12 T cell therapies, and use thereof for the treatment of cancer.

### 2. Description of Related Art

Autologous tumor-infiltrating lymphocyte (TIL) infusion has been a remarkable breakthrough in the treatment of patients with refractory melanoma and has resulted in higher response rates than has BRAF-targeted therapy or CTLA-4-blocking therapy. Most patients should experience a response to TIL transfer because TILs can be isolated from their tumors. However, in practice, the response rates are only about 50%, including a 10%-15% complete response rate (Besser et al., 2010; Radvanyi et al., 2012; Dudley et al., 2005).

Major challenges in TIL therapy are the reduced tumor homing ability of TILs after reinfusion as well as the changes in the tumor microenvironment. In recent clinical trials, 1.5-2×10¹¹ TILs were infused to ensure enough tumor-targeting TILs and successful tumor remission (Radvanyi et al., 2012; Dudley et al., 2005). However, transferring such large numbers of TILs into cancer patients can cause off-target adverse effects. Approaches are needed that enable TILs to be delivered to tumor sites more efficiently and therefore require much smaller numbers of infused T cells.

One reason that TILs cannot reach tumor sites is the loss of tumor homing characteristics during ex vivo culture; thus, new therapies use T cells that have been engineered with receptors that recognize tumor antigens (e.g., CD19), known as chimeric antigen receptor (CAR)-T cell therapy. CAR-T cell therapy more specifically targets tumor cells and has had substantial success in treating hematologic malignancies, in which CAR-T cells target tumor cells in the blood and bone marrow. However, the efficacy of CAR-T cell therapy is limited in solid tumors. Common antigens are lacking on solid tumor cells due to their heterogeneity. In addition, the host conditioning often avoids T cells entering the tumor stroma.

There are multiple challenges for using T cell therapy including CAR-T, TIL, and TCR-T (CTL) cells to treat solid tumors including tumor heterogeneity to escape the antigen or target specific T cell attack, T cell penetration into solid tumors, inactivation of the infiltrated T cells by the immune suppressive environment, and the exhaustion of effector T cells. Thus, there is an unmet need for T cell therapies that are able to penetrate deep into solid tumors.

### SUMMARY

In a first embodiment, the present disclosure provides a construct encoding a tumor-targeted and membrane-anchored interleukin 12 (IL-12). The IL-12 may comprise an IL-12 alpha subunit p35 and an IL-12 beta subunit p40.

In some aspects, the p35 subunit is fused to a transmembrane domain (TM), such as an EGFR transmembrane domain. In certain aspects, the p35/TM subunit is further fused to a signaling domain (SD). For example, the signaling domain is a CD3ζ, CD28, and/or 4-1BB signaling domain. In particular aspects, the signaling domain comprises CD3ζ and 4-1BB signaling domains. In some aspects, the signaling domain is 4-1BB.

In certain aspects, the p40 subunit is fused to the tumor-targeting moiety. For example, the tumor-targeting moiety is a peptide, antibody or fragment thereof. In some aspects, the antibody or fragment thereof is selected from the group consisting of F(ab′)2, Fab′, Fab, Fv, and scFv. In particular aspects, the antibody or fragment thereof is an scFv. In some aspects, the tumor-targeting moiety is a peptide. In specific aspects, the tumor-targeting moiety specifically binds cell surface vimentin (CSV), such as a CSV peptide. In other aspects, the p40 subunit is fused to a transmembrane domain and/or a signaling domain and the p35 subunit is fused to a tumor-targeting moiety. The heterodimer comprising a p35 fusion subunit (p35/TM/SD) and p40 fusion (p40-tumor-targeted moiety) is referred to herein as a chimeric antigen receptor (CAR)-like IL12 (CARL-IL12).

In some aspects, the construct is a viral vector. For example, the viral vector is a retroviral vector or lentiviral vector.

In another embodiment, there is provided a host cell engineered to express the construct of the embodiments (e.g, a construct expressing tumor-targeted and membrane-anchored IL-12 or CARL-IL12). In some aspects, the host cell is an immune cell. For example, the immune cell is a tumor-homing cell. In certain aspects, the immune cell is a T cell. In some aspects, the T cell is a peripheral blood T cell. In some aspects, the T cell is a CD4⁺ T cell or CD8⁺ T cell. In certain aspects, the T cell is autologous or allogeneic. In other aspects, the immune cell is a NK cell.

Further provided herein is a pharmaceutical composition comprising IL-12 immune cells of the embodiments (e.g., an immune cell engineered to express tumor-targeted and membrane-anchored IL-12 or CARL-IL12) and a pharmaceutical carrier. Another embodiment provides a composition comprising an effective amount of IL-12 immune cells of the embodiments (e.g., an immune cell engineered to express a construct expressing membrane-anchored IL-12) for the treatment of cancer in a subject.

In yet a further embodiment, there is provided a method for treating cancer in a subject comprising administering an effective amount of IL-12 immune cells of the embodiments (e.g., an immune cell engineered to express a tumor-targeted and membrane-anchored IL-12 or CARL-IL12 gene) to the subject. In particular aspects, the CARL-IL12 is anchored to the membrane of said immune cells.

In some aspects, the cancer is glioblastoma, cervical cancer, pancreatic cancer, ovarian cancer, uterine cancer, esophageal cancer, melanoma cancer, head and neck cancer, colorectal cancer, bladder cancer, lung cancer, prostate cancer, sarcoma cancer, breast cancer, liver cancer, renal cancer or acute myelogenous leukemia.

In additional aspects, the method further comprises administering at least a second anticancer therapy to the subject. In some aspects, the second anticancer therapy is a surgical therapy, chemotherapy, radiation therapy, cryotherapy, hormonal therapy, immunotherapy or cytokine therapy. In particular aspects, the second anticancer therapy is chemotherapy. In some aspects, the chemotherapy is cyclophosphamide, methotrexate, fluorouracil, doxorubicin, vincristine, ifosfamide, cisplatin, gemcytabine, busulfan, or ara-C. In specific aspects, the chemotherapy is doxorubicin. In some aspects, the chemotherapy is administered prior to the IL-12 (e.g., CARL-IL12) immune cells. In particular aspects, the chemotherapy is administered 24-48 hours prior to the IL-12 (e.g., CARL-IL12) immune cells. In certain aspects, the chemotherapy is administered 15-25 hours prior to the IL-12 (e.g., CARL-IL12) immune cells. In some aspects, administering the IL-12 (e.g., CARL-IL12) immune cells does not induce endogenous IL-12 secretion and/or IFNγ release. In certain aspects, the T cells and/or at least one additional therapeutic agent is administered intravenously, intraperitoneally, intratracheally, intratumorally, intramuscularly, endoscopically, intralesionally, percutaneously, subcutaneously, regionally, or by direct injection or perfusion. In specific aspects, administration of the IL-12 (e.g., CARL-IL12) T cells does not induce IFNγ or induces a lower level of IFNγ as compared to administration of T cells with wild-type IL-12. In some aspects, the IFNγ is measured in a serum sample. In particular aspects, the T cells and/or the second anticancer therapy is administered more than once. In some aspects, the T cells penetrate to or near the center of a tumor within the subject.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Tumor volume of mice treated with CARL-IL12 T cell therapy (labeled as attIL12BBT) alone or in combination with doxorubicin.

FIG. 2: Tumor volume of mice with epithelial tumors treated with CARL-IL12 T cells.

FIG. 3: Side-by-side comparison between CARL-IL12 (ATTIL12BB) modified T cell therapy and unmodified T cell therapy in a human osteosarcoma model in presence of pre-doxorubicin treatment. Osteosarcoma with a size of 1000 mm3 were randomly assigned different treatments as detailed below. Doxorubicin (Dox) was administered. 2 days ahead of T cell therapy via IP at a dose of 1 mg/kg. T cells were administered via i.v route at a dose of 2.5×10E6 for each administration. Two independent administrations were performed against each tumor-bearing mouse. Notx, no treatment; Doxorubicin, Doxorubicin treatment only; CtrlT, unmodified T cells administration only; CtrlT+Dox, unmodified T cells plus doxorubicin were used; ATTIL12T+Dox, T cells modified with tumor-targeted and membrane-anchored IL12 plus doxorubicin were used; ATTIL12BBT+Dox, T cells modified with CARL-IL12 plus doxorubicin treatment; ttIL12T+Dox, T cells modified with tumor-targeted IL12 plus doxorubicin treatment; wtIL12T+Dox, T cells modified with wildtype IL12 plus doxorubicin treatment; anIL12T+Dox, T cells modified with membrane anchored wildtype IL12 plus doxorubicin treatment.

FIG. 4: Side-by-side comparison between CARL-12 T (ATTIL12BBT) cell therapy and other possible IL12-modified T cell therapies in a human osteosarcoma model (see FIG. 3 legend).

FIG. 5: Tumor volume of mice bearing patient-derived xenografts of telangiectatic osteocarcinoma (OS) tumors. The mice were treated with doxorubicin, control T cells, or a combination of T cells and doxorubicin. The T cells were CAR IL-12 T cells (top) or T cells with membrane-anchored IL-12 (bottom).

FIG. 6: Schematic depicting various IL-12 constructs including CARL-IL12 constructs.

FIG. 7: Schematic depicting CARL-IL12 at cell membrane.

FIG. 8: T cell survival on day 19 in basic culture medium without cytokines or antibodies.

FIG. 9: Tumor volume of mice treated with ATT-IL-12 with tumor targeting moiety and not an intracellular signaling component.

### DESCRIPTION OF ILLUSTRATIVE EMBODIMENTS

In certain embodiments, the present disclosure provides a CAR-like construct with membrane-anchored and/or tumor-targeted IL-12. The construct may comprise a tumor-targeting moiety, such as a peptide, antibody, or fragment thereof. An exemplary tumor-targeting moiety is the cell surface vimentin (CSV) peptide or scFv. The construct may be a retroviral vector or lentiviral vector. Further provided herein are cells engineered to express the CARL-IL12 vector, such as immune cells, particularly T cells. The CARL IL-12 T cells may be used to treat a disease or disorder, such as a solid tumor or blood cancer.

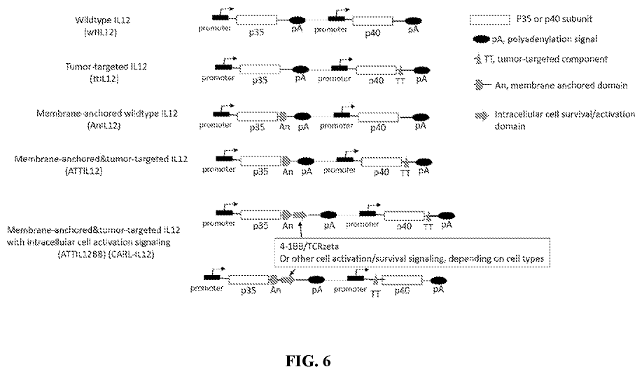

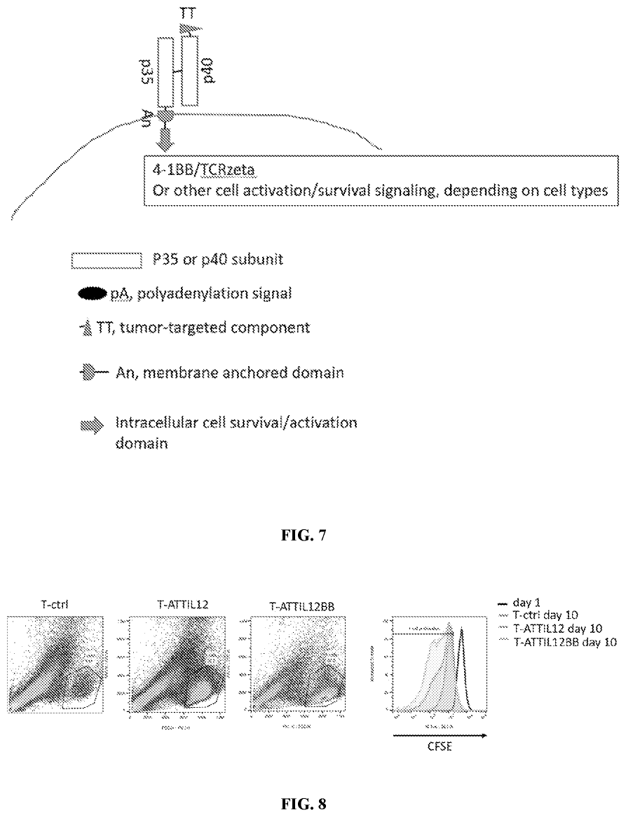

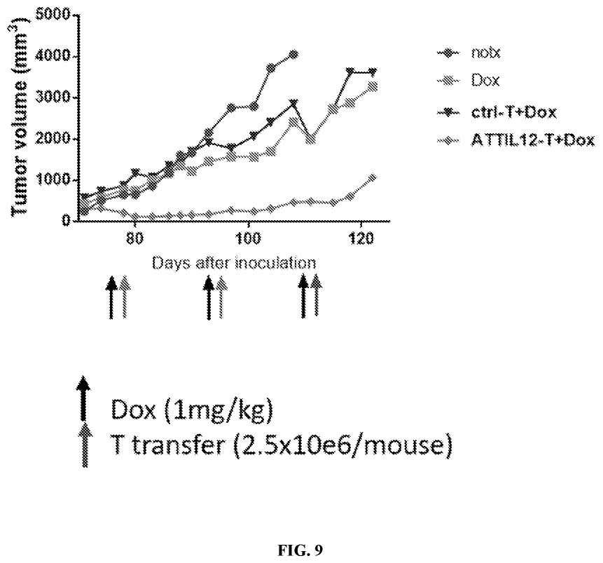

Specifically, the present construct may be CARL-IL-12 construct expressing tumor-targeted and membrane-anchored interleukin 12 (IL-12) with an intracellular cell activation/survival domain (FIGS. 6, 7). The IL-12 may comprise an IL-12 alpha subunit p35 fused with a cell membrane anchoring domain with or without a cell survival/activation domain and an IL-12 beta subunit p40 fused with tumor-targeted peptides. In the present studies, the tumor-targeting moiety alone (ATT-IL-12) without the cell survival/activation domain showed increased anti-tumor efficacy (FIG. 9). Both the CARL-IL12 and the ATT-IL-constructs resulted in increased host T cell proliferation.

Specifically, the IL-12 may comprise both p35 and p40 subunits. The p40 subunit may be fused to the tumor-targeting moiety, such as the CSV peptide. The p35 subunit may be fused to a transmembrane domain, such as EGFR. The p35 subunit may further be fused to a cell signaling domain, such as 4-1BB and CD3. The two subunits together can form the CARL-IL12 construct. The construct can target tumors directly using the tumor-targeted peptide or scFV in the p40 subunit and induces T cell proliferation through the membrane anchored p35-TM-4-1BB or the p35-TM (i.e., without 4-1BB) subunit. The 4-1BB can be replaced with other cell activation/survival signaling domains depending on the cell type. As a result, the CARL-IL12 can be used for treating large tumors, such as drug-resistant sarcomas. The CARL-IL12 can further reduce the toxicity concerns from CAR T cell therapy and IL-12 therapy by targeting T cells rapidly to tumors. The CARL-IL12 therapy may be administered in combination with chemotherapy, such as doxorubicin, and it may boost tumor-specific TCR-T cell induction. The CARL-IL12 T cell therapy can also reduce cytokine release syndrome. Indeed, the present studies showed that the present CARL-IL12 therapy had superior anti-tumor efficacy as compared to wildtype IL-12 T cell therapy and membrane anchored IL-12 T cell therapy.

Methods are also provided for the isolation of T cells from the blood of a subject, modification with CARL-IL12, expansion, and administration to the subject. In addition, subjects may be pretreated with doxorubicin or other T cell recruiting inducers.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “membrane-anchored IL-12” refers to an IL-12 protein that comprises a transmembrane domain (FIG. 6). The term “membrane-anchored tumor-targeted IL-12 (attIL-12)” refers to an IL-12 protein that comprises both a transmembrane domain and a tumor-targeted domain (e.g., FIG. 6).

A polynucleotide or polynucleotide region (or a polypeptide or polypeptide region) has a certain percentage (for example, 80%, 85%, 90%, or 95%) of “percent similarity” or “sequence similarity” which refers to the degree by which one amino acid may substitute for another amino acid without loss of function. This percent similarity can be determined through the use of a matrix such as the PAM250 or BLOSUM62 matrix.

A polynucleotide or polynucleotide region (or a polypeptide or polypeptide region) has a certain percentage (for example, 80%, 85%, 90%, or 95%) of “sequence identity” or “homology” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60, expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

### II. CAR-LIKE IL-12 (CARL-IL12) T CELL THERAPY

Certain embodiments of the present disclosure concern a CAR-like construct with membrane-anchored IL-12. In some aspects, the construct or expression vector is a retroviral expression vector, an adenoviral expression vector, a DNA plasmid expression vector, or an AAV expression vector. The construct may be a viral vector, such as a retroviral vector or lentiviral vector. Specifically, the IL-12 may comprise both p35 and p40 subunits. The p40 subunit may be fused to the tumor-targeting moiety. The p35 subunit may be fused to a transmembrane domain. The p35 subunit may further be fused to a cell signaling domain. The signaling domain may be CD3, CD28, and/or 4-1BB signaling domains. The two fusion subunits together can form the CARL-IL12 construct.

The construct may comprise a tumor-targeting moiety, such as a peptide, antibody, or fragment thereof. The tumor-targeting moiety may be the antigen-binding portion or portions of an antibody molecule, such as a single-chain antibody fragment (scFv) derived from the variable heavy (VH) and variable light (VL) chains of a monoclonal antibody (mAb).

An exemplary tumor-targeting moiety is the cell surface vimentin (CSV) peptide or scFv. Beside the induced NKG2D ligand target in tumors via this CARL-IL12 T cell therapy plus pre-chemotherapy such as doxorubicin, the second universal tumor-specific target is cell surface vimentin (CSV). CSV is detected across any types of highly malignant tumors and is primarily found on highly malignant tumors such as metastatic and relapsed tumors. For example, studies have shown that CSV was present on 100% metastatic tumor cell surfaces of colon tumors and 97-98% of drug or CAR-T cell resistant or relapsed ALL.

The CARL-IL12 construct may comprise a transmembrane domain, such as to anchor the antibody to a cell. Any transmembrane domain known in the art may be used for the membrane-anchored expression of the CARL-IL12 to the host cell, such as T cells. An exemplary transmembrane domain is the EGFR transmembrane domain. In other embodiments, the transmembrane domain may comprise other transmembrane sequence known in the art such as disclosed in Kozma et al., Nucleic Acids Research 41 Database Issue, D524-D529, 2013. In other embodiments, the IL-12 p35 comprises a transmembrane domain. Well known examples of transmembrane proteins having one or more transmembrane polypeptide domains include members of the integrin family, CD44, glycophorin, MHC Class I and II glycoproteins, EGF receptor, G protein coupled receptor (GPCR) family, receptor tyrosine kinases (such as insulin-like growth factor 1 receptor (IGFR) and platelet-derived growth factor receptor (PDGFR)), porin family and other transmembrane proteins. Certain embodiments of the present disclosure contemplate using a portion of a transmembrane polypeptide domain such as a truncated polypeptide having membrane-inserting characteristics as may be determined according to standard and well known methodologies.

The membrane-anchored IL-12 protein sequences that can be used in various embodiments include the amino acid sequences of wild-type IL-12, as well as analogues and derivatives thereof. The analogues and derivatives can include, but are not limited to, additions or substitutions of amino acid residues within the amino acid sequences encoded by a nucleotide sequence, but that result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Amino acid substitutions may alternatively be made on the basis of the hydropathic index of amino acids. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The use of the hydropathic amino acid index in conferring interactive biological function on a protein is understood in the art (Kyte and Doolittle, J. Mol. Biol. 157:105-132, 1982). It is known that in certain instances, certain amino acids may be substituted for other amino acids having a similar hydropathic index or score and still retain a similar biological activity. In making changes based upon the hydropathic index, in certain embodiments the substitution of amino acids whose hydropathic indices are within ±2 is included, while in other embodiments amino acid substitutions that are within ±1 are included, and in yet other embodiments amino acid substitutions within ±0.5 are included.

Amino acid substitutions may alternatively be made on the basis of hydrophilicity, particularly where the biologically functional protein or peptide thereby created is intended for use in immunological embodiments. In certain embodiments, the greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments the substitution of amino acids whose hydrophilicity values are within ±2 is included, in certain embodiments those that are within ±1 are included, and in certain embodiments those within ±0.5 are included. One may also identify epitopes from primary amino acid sequences on the basis of hydrophilicity.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

A. T Cell Preparation

Further provided herein are cells engineered to express the CARL-IL12 vector, such as immune cells, particularly T cells. The CARL-IL12 T cells may be used to treat a disease or disorder, such as a solid tumor or blood cancer.

Certain embodiments of the present disclosure concern obtaining a starting population of T cells, modifying the T cells, and administering the modified T cells to a subject as an immunotherapy to target cancer cells. In particular, the T cells express CARL-IL12. Several basic approaches for the derivation, activation and expansion of functional anti-tumor effector T cells have been described in the last two decades. These include: autologous cells, such as tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods described herein.

In some embodiments, the starting population of T cells are derived from the blood, bone marrow, lymph, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days. For example, the cells may be cultured from 5 days, 5.5 days, or 5.8 days to 21 days, 21.5 days, or 21.8 days, such as from 10 days, 10.5 days, or 10.8 days to 14 days, 14.5 days, or 14.8 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15). The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as a human leukocyte antigen A2 (HLA-A2) binding peptide, in the presence of a T-cell growth factor, such as 300 IU/ml IL-2. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2⁺ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

B. T Cell Activation

In some embodiments, the present disclosure provides methods of activating T cells to increase expression of NKG2D receptor on the T cells, such as CD8⁺ T cells. The starting population of T cells may be pre-treated with anti-CD3, such anti-CD3 beads. The pre-treatment may be for about 12 hours to 3 days, such as about 24 hours. The expanded T cells may then be cultured with CD80 protein, such as CD80-Fc recombinant protein to induce CD28 activation and, thus, NKG2D expression. The culture with CD80 may be for about 1-6 days, such as about 1, 2, 3, 4, 5, or 6 days, particularly about 4 days. In some aspects, the T cells may be treated with anti-CD3 and CD80 simultaneously.

C. Genetically Engineered T Cells

The T cells of the present disclosure can be genetically engineered to express the present CARL IL-12 construct. The construct may comprise an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

In some aspects, the antigen-specific binding, or recognition component is fused to p40 subunit, which physiologically attached to p35-fusion subunit extracellularly to form a heterodimer, which becomes a CARL-structure with p35-fusion in charge of intracellular signaling and with p40-fusion subunit in charge of antigen-specific binding.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, CD 154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

The CARL-IL12 generally includes at least one intracellular signaling component or components. In some embodiments, the CARL-IL12 includes an intracellular component of the TCR complex, such as a TCR CD3⁺ chain that mediates T-cell activation and cytotoxicity, e.g., CD3 zeta chain. Thus, in some aspects, the antigen binding molecule is linked to one or more cell signaling modules. In some embodiments, cell signaling modules include CD3 transmembrane domain, CD3 intracellular signaling domains, and/or other CD transmembrane domains. In some embodiments, the CAR further includes a portion of one or more additional molecules such as Fc receptor γ, CD8, CD4, CD25, or CD16. For example, in some aspects, the CARL-IL12 includes a chimeric molecule between CD3-zeta (CD3-Q or Fc receptor γ and CD8, CD4, CD25 or CD16.

D. Methods of Delivery

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

a. Viral Vectors

Viral vectors encoding a CARL may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

b. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

(i) Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp-upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

(ii) Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth diease virus 2A) or a “2A-like” sequence (e.g., Thosea asigna virus 2A; T2A).

(iii) Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

c. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

d. Other Methods of Nucleic Acid Delivery

In addition to viral delivery of the nucleic acids encoding the antigen receptor, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

### III. METHODS OF TREATMENT

Further provided herein are methods for treating or delaying progression of cancer in an individual comprising administering to the individual an effective amount a CARL IL-12 T cell therapy. Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

In some embodiments, the individual has cancer that is resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

In some embodiments, the subject is administered a chemotherapeutic in combination with the T cell therapy. For example, the chemotherapeutic may be doxorubicin (Dox) or cyclophosphamide. Subjects may be pretreated with chemotherapeutic such as doxorubicin or other T cell recruiting inducers. The pretreatment may be 16-24 hours prior to the T cell therapy.

In some embodiments, T cells are autologous. However, the cells can be allogeneic if the endogenous TCRs are knockout. In some embodiments, the T cells are isolated from the patient themself, so that the cells are autologous. If the T cells are allogeneic, the endogenous TCR needs to be removed. The cells are administered to the subject of interest in an amount sufficient to control, reduce, or eliminate symptoms and signs of the disease being treated.

The effectiveness of treatment can be measured by many methods known to those of skill in the art. In one embodiment, a white blood cell count (WBC) is used to determine the responsiveness of a subject's immune system. A WBC measures the number of white blood cells in a subject. Using methods well known in the art, the white blood cells in a subject's blood sample are separated from other blood cells and counted. Normal values of white blood cells are about 4,500 to about 10,000 white blood cells/μl. Lower numbers of white blood cells can be indicative of a state of immunosuppression in the subject.

In another embodiment, immunosuppression in a subject may be determined using a T-lymphocyte count. Using methods well known in the art, the white blood cells in a subject's blood sample are separated from other blood cells. T-lymphocytes are differentiated from other white blood cells using standard methods in the art, such as, for example, immunofluorescence or FACS. Reduced numbers of T cells, or a specific population of T-cells can be used as a measurement of immunosuppression. A reduction in the number of T cells, or in a specific population of T cells, compared to the number of T cells (or the number of cells in the specific population) prior to treatment can be used to indicate that immunosuppression has been induced.

In additional embodiments, tests to measure T cell activation, proliferation, or cytokine responses including those to specific antigens are performed. In some examples, the number of Treg or Breg cells can be measured in a sample from a subject. In additional examples, cytokines are measured in a sample, from a subject, such as IL-10.

In other examples, to assess inflammation, neutrophil infiltration at the site of inflammation can be measured. In order to assess neutrophil infiltration myeloperoxidase activity can be measured. Myeloperoxidase is a hemoprotein present in azurophilic granules of polymorphonuclear leukocytes and monocytes. It catalyzes the oxidation of halide ions to their respective hypohalous acids, which are used for microbial killing by phagocytic cells. Thus, a decrease in myeloperoxidase activity in a tissue reflects decreased neutrophil infiltration, and can serve as a measure of inhibition of inflammation.

In another example, effective treatment of a subject can be assayed by measuring cytokine levels in the subject. Cytokine levels in body fluids or cell samples are determined by conventional methods. For example, an immunospot assay, such as the enzyme-linked immunospot or “ELISPOT” assay, can be used. The immunospot assay is a highly sensitive and quantitative assay for detecting cytokine secretion at the single cell level. Immunospot methods and applications are well known in the art and are described, for example, in Czerkinsky et al., 1988; Olsson et al., 1990; and EP 957359. Variations of the standard immunospot assay are well known in the art and can be used to detect alterations in cytokine production in the methods of the disclosure (see, for example, U.S. Pat. Nos. 5,939,281 and 6,218,132).

In some embodiments, the subject can be administered nonmyeloablative lymphodepleting chemotherapy prior to the T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a T cell growth factor that promotes the growth and activation of the autologous T cells is administered to the subject either concomitantly with the autologous T cells or subsequently to the autologous T cells. The T cell growth factor can be any suitable growth factor that promotes the growth and activation of the autologous T-cells. Examples of suitable T cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2. IL-12 is a preferred T-cell growth factor.

Local, regional or systemic administration may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

B. Pharmaceutical Compositions

Also provided herein are pharmaceutical compositions and formulations comprising a T cell therapy and a pharmaceutically acceptable carrier.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22nd edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

C. Additional Therapy

In certain embodiments, the compositions and methods of the present embodiments involve a CARL-IL12 T cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

A T cell therapy may be administered before, during, after, or in various combinations relative to an additional therapy, such as doxorubicin. The administrations may be in intervals ranging from concurrently to minutes to days to weeks. In embodiments where the T cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the T cell therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

The T cell therapy and the additional therapeutic agent may be administered by the same route of administration or by different routes of administration. In some embodiments, the T cell therapy and/or anti-platelet agent is administered intravenously, intramuscularly, subcutaneously, topically, orally, transdermally, intraperitoneally, intraorbitally, by implantation, by inhalation, intrathecally, intraventricularly, or intranasally. An effective amount of the T cell therapy and additional therapeutic agent may be administered for prevention or treatment of disease. The appropriate dosage of the T cell therapy and additional therapeutic agent be determined based on the type of disease to be treated, severity and course of the disease, the clinical condition of the individual, the individual's clinical history and response to the treatment, and the discretion of the attending physician.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

Various combinations may be employed. For the example below a T cell therapy is “A” and an additional therapeutic agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaIl); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody—drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen (Carter et al., 2008; Teicher et al., 2014; Leal et al., 2014). Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach (Teicher et al., 2009) and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints are regulators in the immune system that either turn up a signal (e.g., co-stimulatory molecules) or turn down a signal. Inhibitory checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. ARTICLES OF MANUFACTURE OR KITS

An article of manufacture or a kit is provided comprising T cells expressing CARL IL-12 is also provided herein. The article of manufacture or kit can further comprise a package insert comprising instructions for using the adoptive T cells optionally in conjunction with an additional therapeutic agent (e.g., doxorubicin) to treat or delay progression of cancer in an individual or to enhance immune function of an individual having cancer. Any of the adoptive T cells and/or additional therapeutic agents described herein may be included in the article of manufacture or kits. In some embodiments, the adoptive T cells and additional therapeutic agent are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Expression of IL-12 in CAR-Like Construct

A chimeric antigen receptor-like construct was developed with membrane-bound IL-12 being fused to a tumor-targeted peptide CSV. In brief, the two subunits of IL12 were cloned into a single vector. The p35 subunit was fused with EGFR transmembrane domain and the 4-1BB encoding sequence in the same reading frame and the p40 subunit was fused with the CSV binding peptide encoding sequence. The CARL-IL12 fusion gene was packaged into a lentiviral vector.

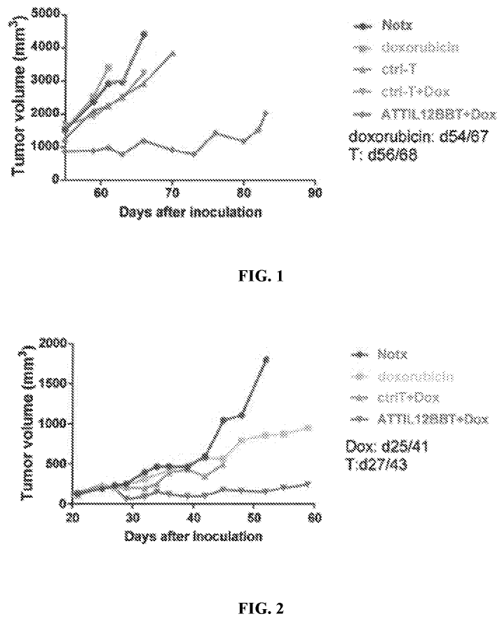

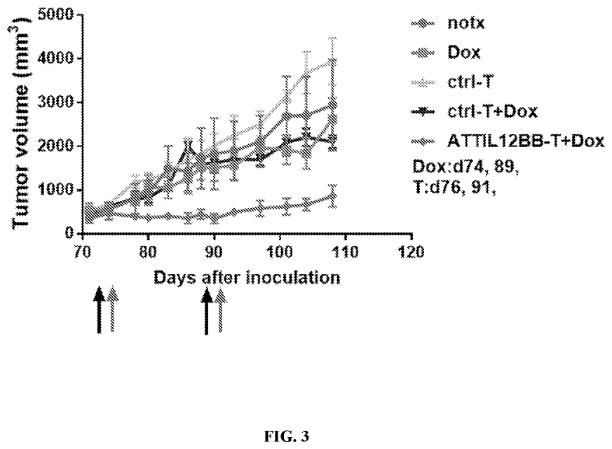

Mice bearing a mesenchymal tumor were used to test the CARL-IL12 therapy. The virus containing the CARL-IL12 fusion gene was transfected into T cells expanded from peripheral blood and administered into tumor bearing mice via tail vein one day after administration of doxorubicin (DOX). The doxorubicin treatment dose was 1 mg/kg one or two days ahead of the different treatments. The other control groups included no treatment (Notx), doxorubicin (Dox) alone, control T cells alone (ctrl-T), and Dox plus control T cells (Ctrl-T). The CARL-IL12 T cell therapy was labeled as attIL12BBT, in which Dox was administered one day ahead of the treatment and a total of two treatments for each mouse was performed. 2.5 million T cells were administered into the treated mice on days 56 and 68 respectively; doxorubicin was administered on days 54 and 67 respectively (FIG. 1). The study was then repeated with doxorubicin administered on days 25 and 41 while T cells were administered on days 27 and 43, respectively (FIG. 2). The mice treated with the CARL-IL12 T cells had a significant reduction in tumor volume as compared to the controls (FIG. 2).

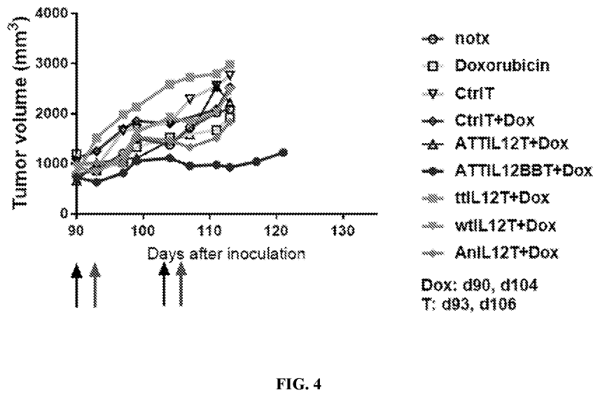

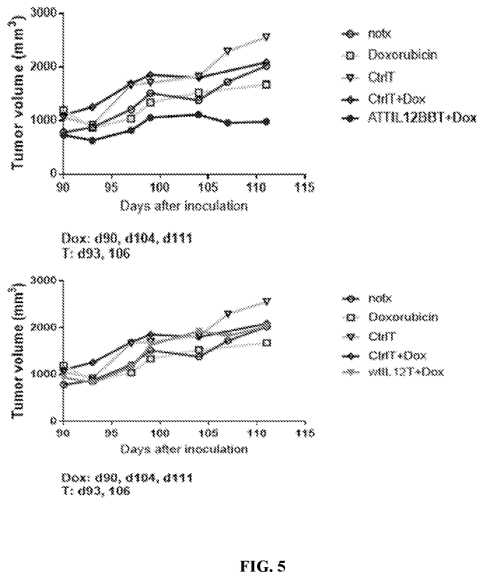

The CAR IL-12 T cells were then compared to T cells with membrane-anchored IL-12 (FIG. 4). Mice were treated with T cells on days 78, 93, and 105 at a dose of 2.5×10⁶ per mouse. Doxorubicin was administered on days 77 and 91 at a dose of 1 mg/kg. It was observed that the mice treated with the present CAR IL-12 T cells and doxorubicin had a lower tumor volume as compared to the mice treated with IL-12 T cells (FIGS. 4-5).

FIG. 6 shows the various IL-12 constructs including the CARL-IL12 construct as well as the ATT-IL-12 construct without the cell intracellular signaling. Both of the constructs enhanced T cells proliferation (FIG. 8). In addition, the ATT-IL-12 construct resulted in a decrease in tumor volume in some tumor model (FIG. 9). Thus, both constructs may be used as therapeutics.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Austin-Ward and Villaseca, Revista Medica de Chile, 126(7):838-845, 1998.
Ausubel et al., Current Protocols in Molecular Biology, Greene Publishing Associates and John Wiley & Sons, N Y, 1994.
Besser et al. Clin Cancer Res. 16(9):2646-55, 2010.
Bukowski et al., Clinical Cancer Res., 4(10):2337-2347, 1998.
Camacho et al. J Clin Oncology 22(145): Abstract No. 2505, 2004.
Carter et al. Cancer J 14(3):154-69, 2008.
Christodoulides et al., Microbiology, 144 (Pt 11):3027-3037, 1998.
Czerkinsky et al., J. Immunol. Methods 1988; 110:29-36.
Davidson et al., J. Immunother 21(5):389-398, 1998.
Dudley et al. Clinical Oncology. 23(10):2346-57, 2005.
European Patent Publication No. EP957359
Hanibuchi et al., Int. J. Cancer, 78(4):480-485, 1998.
Hellstrand et al., Acta Oncologica, 37(4):347-353, 1998.
Hollander, Front. Immun., 3:3, 2012.
Hui and Hashimoto, Infection Immun., 66(11):5329-5336, 1998.
Hurwitz et al. Proc Natl Acad Sci USA 95(17): 10067-10071, 1998.
International Patent Publication No. WO2014/05566
International Patent Publication No. WO1995001994
International Patent Publication No. WO1998042752
International Patent Publication No. WO2000037504
International Patent Publication No. WO2001014424
International Patent Publication No. WO2014/055668
International Patent Publication No. WO2014031687
International Patent Publication No. WO2015016718
Kozma et al., Nucleic Acids Research 41 Database Issue, D524-D529, 2013.
Kyte and Doolittle, J. Mol. Biol. 157:105-132, 1982.
Leal et al., Ann N Y Acad Sci. 2014; 1321:41-54, 2014.
Li Nat Biotechnol. 23:349-354, 2005.
Mokyr et al. Cancer Res 58:5301-5304, 1998.
Olsson et al. J. Clin. Invest. 1990; 86:981-985.
Pardoll Nat Rev Cancer, 12(4): 252-64, 2012.
Qin et al., Proc. Natl. Acad. Sci. USA, 95(24):14411-14416, 1998.
Radvanyi et al. Clin Cancer Res. 18(24):6758-70, 2012.
Sambrook et al., Molecular Cloning: A Laboratory Manual, 3^{rd} ed., Cold Spring Harbor Press, Cold Spring Harbor, N.Y. 2001.
Teicher B A. Current cancer drug targets. 9(8):982-1004, 2009.
Teicher B A. Current opinion in oncology. 26(5):476-83, 2014.
Terakura et al. Blood. 1:72-82, 2012.
U.S. Pat. No. 4,870,287
U.S. Pat. No. 4,897,355
U.S. Pat. No. 4,946,787
U.S. Pat. No. 5,049,386
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,844,905
U.S. Pat. No. 5,846,945
U.S. Pat. No. 5,885,796
U.S. Pat. No. 5,939,281
U.S. Pat. No. 6,207,156
U.S. Pat. No. 6,218,132
U.S. Pat. No. 6,225,042
U.S. Pat. No. 6,355,479
U.S. Pat. No. 6,362,001
U.S. Pat. No. 6,410,319
U.S. Pat. No. 6,451,995
U.S. Pat. No. 6,790,662
U.S. Pat. No. 7,070,995
U.S. Pat. No. 7,265,209
U.S. Pat. No. 7,354,762
U.S. Pat. No. 7,446,179
U.S. Pat. No. 7,446,190
U.S. Pat. No. 7,446,191
U.S. Pat. No. 8,008,449
U.S. Pat. No. 8,017,114
U.S. Pat. No. 8,119,129
U.S. Pat. No. 8,252,592
U.S. Pat. No. 8,324,353
U.S. Pat. No. 8,329,867
U.S. Pat. No. 8,339,645
U.S. Pat. No. 8,354,509
U.S. Pat. No. 8,398,282
U.S. Pat. No. 8,479,118
U.S. Pat. No. 8,735,553
U.S. Patent Publication No. 2005/0260186
U.S. Patent Publication No. 2006/0104968
U.S. Patent Publication No. US20110008369
U.S. Patent Publication No. US2014022021
U.S. Patent Publication No. US20140294898
Wang et al. J Immunother 35(9):689-701, 2012.

What is claimed is:

1. A construct encoding a tumor-targeted and membrane-anchored IL-12.

2. The construct of claim 1, wherein the tumor-targeted and membrane-anchored IL-12 comprises an IL-12 p35 subunit and an IL-12 p40 subunit.

3. The construct of claim 1 or 2, wherein the IL-12 p35 encoding DNA is fused to a transmembrane domain encoding DNA in the same reading frame.

4. The construct of claim 3, wherein the transmembrane domain is an EGFR transmembrane domain.

5. The construct of any of claims 1-4, wherein the p35 subunit is linked to a signaling domain encoding sequence.

6. The construct of claim 5, wherein the signaling domain is a CD3, CD28, and/or 4-1BB signaling domain.

7. The construct of claim 5, wherein the signaling domain comprises CD3 and 4-1BB signaling domains.

8. The construct of claim 5, wherein the signaling domain is 4-1BB.

9. The construct of any of claims 1-8, wherein the p40 subunit encoding DNA is fused to the tumor-targeting moiety encoding DNA in the same reading frame.

10. The construct of claim 9, wherein tumor-targeting is achieved by a tumor-targeting moiety comprising a peptide, antibody or fragment thereof.

11. The construct of claim 10, wherein the antibody or fragment thereof is selected from the group consisting of F(ab′)2, Fab′, Fab, Fv, and scFv.

12. The construct of claim 10, wherein the antibody or fragment thereof is an scFv.

13. The construct of claim 10, wherein the tumor-targeting moiety comprises is a peptide.

14. The construct of any of claims 1-10, wherein the tumor-targeting IL-12 specifically binds cell surface vimentin (CSV).

15. The construct of claim 13, wherein the tumor-targeting moiety is a CSV peptide.

16. The construct of any of claims 1-15, wherein the construct is a viral vector.

17. The construct of claim 16, wherein the viral vector is a lentiviral vector.

18. A host cell engineered to express the construct of any of claims 1-17.

19. The host cell of claim 18, wherein the host cell is an immune cell.

20. The host cell of claim 19, wherein the immune cell is a tumor-homing cell.

21. The host cell of claim 19, wherein the immune cell is a T cell.

22. The host cell of claim 21, wherein the T cell is a peripheral blood T cell.

23. The host cell of claim 21, wherein the T cell is a CD4⁺ T cell or CD8⁺ T cell.

24. The host cell of claim 21, wherein the T cell is autologous.

25. The host cell of claim 21, wherein the T cell is allogeneic.

26. The host cell of claim 19, wherein the immune cell is a NK cell.

27. A pharmaceutical composition comprising IL-12 immune cells of any of claims 18-26 and a pharmaceutical carrier.

28. A composition comprising an effective amount of tumor-targeted IL-12 immune cells of any of claims 18-26 for use in the treatment of cancer in a subject.

29. A method for treating cancer in a subject comprising administering an effective amount of immune cells of any of claims 18-26 to the subject.

30. The method of claim 29, wherein the tumor-targeted IL-12 is anchored to the membrane of said immune cells.

31. The method of claim 29, wherein the cancer is glioblastoma, cervical cancer, pancreatic cancer, ovarian cancer, uterine cancer, esophageal cancer, melanoma cancer, head and neck cancer, colorectal cancer, bladder cancer, lung cancer, prostate cancer, sarcoma cancer, breast cancer, liver cancer, renal cancer or acute myelogenous leukemia.

32. The method of any of claims 29-31, further comprising administering at least a second anticancer therapy to the subject.

33. The method of claim 32, wherein the second anticancer therapy is a surgical therapy, chemotherapy, radiation therapy, cryotherapy, hormonal therapy, immunotherapy or cytokine therapy.

34. The method of claim 32, wherein the second anticancer therapy is chemotherapy.

35. The method of claim 34, wherein the chemotherapy is cyclophosphamide, methotrexate, fluorouracil, doxorubicin, vincristine, ifosfamide, cisplatin, gemcytabine, busulfan, or ara-C.

36. The method of claim 34, wherein the chemotherapy is doxorubicin.

37. The method of claim 34 or 36, wherein the chemotherapy is administered prior to the IL-12 immune cells.

38. The method of claim 37, wherein the chemotherapy is administered 24-48 hours prior to the IL-12 immune cells.

39. The method of claim 37, wherein the chemotherapy is administered 15-25 hours prior to the IL-12 immune cells.

40. The method of any of claims 29-38, wherein administering the IL-12 immune cells does not induce endogenous IL-12 secretion and/or IFNγ release.

41. The method of any of claims 29-38, wherein the method induces endogenous tumor-specific T cell expansion and tumor killing.

42. The method of claim 58, wherein the T cells and/or at least one additional therapeutic agent is administered intravenously, intraperitoneally, intratracheally, intratumorally, intramuscularly, endoscopically, intralesionally, percutaneously, subcutaneously, regionally, or by direct injection or perfusion.

43. The method of claim 48, wherein administration of the IL-12 T cells does not induce IFNγ or induces a lower level of IFNγ as compared to administration of T cells with wild-type IL-12.

44. The method of claim 43, wherein the IFNγ is measured in a serum sample.

45. The method of claim 58, wherein the T cells and/or the second anticancer therapy is administered more than once.

46. The method of claim 58, wherein the T cells penetrate to or near the center of a tumor within the subject.
